(19) United States
(12) Patent Application Publication
(10) Pub. No.: US 2017/0065184 A1
(43) Pub. Date: Mar. 9, 2017

(54) SYSTEMS AND METHODS FOR CONTACTLESS ARTERIAL PRESSURE ESTIMATOR

(71) Applicant: SENSIFREE LTD., Kfar Saba (IL)

(72) Inventor: Ilan BARAK, Kfar Saba (IL)

(73) Assignee: SENSIFREE LTD., Kfar Saba (IL)

(21) Appl. No.: 14/893,045

(22) PCT Filed: Jul. 10, 2015
(86) PCT No.: PCT/IB15/55231

§ 371 Date: Nov. 14, 2016

Related U.S. Application Data
(60) Provisional application No. 62/024,403, filed on Jul. 14, 2014.

(51) Int. Cl. A61B 5/021; A61B 5/00

(52) U.S. Cl. CPC A61B 5/021; A61B 5/681; A61B 5/7257; A61B 5/725; A61B 2560/0223; A61B 2562/0228

(57) ABSTRACT

Methods, apparatuses, devices and systems for measuring the arterial blood pressure in humans and mammals by estimating the time varying arterial diameter using electromagnetic fields in the microwave spectrum (for example), are disclosed. Embodiments may be suitable for wearable devices, and for use by medical practitioners.

### FIELD OF THE INVENTION

The invention relates to blood pressure measurement.

Embodiments of the present disclosure provide methods, apparatuses, devices and systems, for measuring the arterial blood pressure in human and mammals. These embodiments estimate the time varying arterial diameter using electromagnetic fields in the microwave spectrum (for example). Such embodiments may be suitable for wearable devices as well as for use by medical practitioners.

### DISCUSSION OF THE BACKGROUND

The sphygmomanometer is currently the most widely used noninvasive apparatus for arterial blood pressure. The detection methods used with this apparatus are auscultatory technique (Riva-Rocci 1896, Korotkoff 1905) and the oscillometric method (Geddes 1970). The auscultatory method by Korotkoff being the golden standard for noninvasive arterial blood measurement.

Methods trying to estimate the arterial blood pressure from the Pulse Wave Velocity, such as “Cuff less Continuous Non-Invasive Blood Pressure Measurement Using Pulse Transit Time Measurement,” by Surendhra Goli, Jayanthi T, (International Journal of Recent Development in Engineering and Technology Website: www.ijrdet.com (ISSN 2347-6435 (Online) Volume 2, Issue 1, January 2014) suggest equations to estimate the Systolic Blood Pressure (SBP) and Diastolic Blood Pressure (DBP) using the Pulse Wave Velocity (PWV) as a single parameter. This approach is flawed for at least two reasons: (1) The PWV depends on the artery tree sections diameters and their flexibility; does not depend on the heart ventricle volume; and it therefore cannot be a single metric to be used to calculate the arterial blood pressure; and (2) using a single parameter to estimate both the SBP and DPB implies that these two are mathematically related, so for any given SBP, the DBP can be calculated; and it is well known that these two values are independent of each other, or else there would be no sense in measuring both of them.

WO/2013/118121 by Barak, incorporated here by reference, teaches a method of estimating the heart rate of a human or animal, using radar means. In this application, in some embodiments, measurement of heart rate does not require calibration of the signal strength to the artery diameter or internal pressure. For such embodiments, the mere frequency of change of these values is sufficient to extract the subject's heart rate. Portions of WO/2013/118121 by Barak are expressly included herein below.

Otto Frank, “Die Grundform des Arteriellen Pulses,” Zeitschrift fur Biologie 37: 483-526 (1899) explained the pulse pressure wave exponential tail mechanism, and his paper is incorporated here by reference.

Conventional photoplethysmogram (PPG) measures changes in optical absorption rates of varying blood volumes, in the skin, up to a few hundreds of microns from the skin surface. The PPG sensor needs to be in tight contact with the skin, and its output signal level is sensitive to the pressure that connects it to the skin. A calibrated measurement of the absolute time varying blood volume quantity in the skin is impractical, due to the uncontrolled changes this pressure as the subject moves. This is especially true if the calibration is performed by changing the limb position.

### SUMMARY OF SOME OF THE EMBODIMENTS

The invention uses electromagnetic radiation transmitted from outside the body of a living being to inside the body, and reflections back to a sensor locating outside the body, for determining artery pressure.

The reflected signal provides a measure of change in diameter of an artery in the body from which some of the electromagnetic radiation is reflected. Reflections of electromagnetic radiation can also be used to remove variations in relative position of the transmitter and sensor, relative to their distance from the skin and the artery, so that the signal can be more representative of variations with time in the diameter of the artery in the body near the sensor.

In one aspect, the invention provides transmitting a modulated microwave signal near the wrist of a person. Artery periodic expansion leads to varying reflected signal strength. A sensor uses reflection from other tissue to compensate for movement of the body of the individual wearing the sensor to the signal resulting from artery periodic expansion. This technique enables a fully electronic-based solution with no mechanical or electro-mechanical components, offering compactness, low cost, and high reliability.

In aspects, the invention uses electromagnetic radiation containing frequencies that can penetrate tissue to a few millimeters; includes electronics that can distinguish different tissue boundaries by time gating; includes a transmitter and sensor that can be positioned up to one centimeter away from the skin. This insensitivity to distance from the skin allows the sensor to be in a wrist band that fits loosely over the wrist, which is a distinct advantage over prior art PPG technology. This insensitivity to distance from the skin allows the sensor to be located adjacent other regions of the body where a firm contact to the skin would not be feasible. For example, the sensor may attached or embedded in an article designed to be worn near any other part of the body having an artery near the skin. These include a femoral artery; a brachial artery; a carotid artery; or a superficial temporal artery. This allows the sensor to be embedded in or attached to headgear, a helmet; a necklace, an ankle bracelet; clothing covering the upper arm; and clothing covering the upper leg; or a wearable strap designed to position the sensor near one of the noted arteries. The term ‘fits loosely’ means that the sensor does not have to be in a secure contact with the skin; and that the structure holding the sensor does not need to maintain tension pressing the sensor against the skin.

In one aspect, a method of the invention provides for calibrating pressure difference to signal level sensitivity. This calibration can be effected by measuring signal average of a sensor worn on a part of the body when that part of the body is at two different heights relative to the height of the hears. For example, a user can lift their wrist a known height when wearing the sensor on a wristband. This calibration may include using a predetermined value for blood specific gravity to calculate a signal ratio due to change in average blood pressure resulting from the change in hydrostatic pressure due to the change in height.

In one aspect, a method of the invention provides for fitting a time segment of sensor values assumed to be proportional to blood pressure, to an exponentially decaying curve. The time segment so fit corresponds to a time during which arterial pressure is falling. that is, a tail, of the pressure wave in the artery. The magnitude of the Systolic and Diastolic pressures can be determined by using equations for the derivative of the exponential curve at different times during the time segment.

In one aspect, a method of the invention provides for correction of the wave shape due to propagation of blood in the artery tree to the location of the sensor. Correction of the wave shape due to propagation of blood in the artery tree may assume a decorrelation function based upon time or frequency response of the arterial tree. Correction of the wave shape due to propagation of blood in the artery tree may be estimating based on either the waveform or the waveform and artery pulse wave velocity.

In some of the embodiments of the present disclosure, an apparatus, device and/or system is provided, which is configured to estimate at least either the difference between the Systolic and Diastolic blood pressure or and preferably the Systolic and Diastolic blood pressure (as would correspond to measured values for such via a sphygmomanometer). The apparatus includes radar means utilizing frequency stepped pulsed compression as explained in “Ultra Wideband Radar Technology” by J Tylor CRC press 2001 which is configured to substantially continually measure (and in some embodiments, continually measure) the cross section of an artery (e.g., the radial artery at the wrist). In some embodiments, the apparatus includes calibration means which may be used to calibrate and estimate one or more blood pressure parameters. Other alternative RADAR methods, for example, chirp or FMCW, may also be used.

In some embodiments, blood pressure is measured by calibrating a radar signal reading difference to a pressure difference. This may be accomplished by measuring the same artery at different positions (for example, with the hand raised and lowered) using the radar means. The unwanted relative movement of the sensor versus the measured artery is compensated, and the absolute Systolic and Diastolic pressure values are estimated by approximating the values and time derivatives of the blood pressure wave.

In some embodiments of the subject disclosure, the measured blood pressure can further be estimated at other body positions, for example in the upper arm Brachial Artery or in the Aorta.

In some embodiments, a blood pressure calculation apparatus configured to calculate blood pressure of a patient based on sensing an artery pressure wave of the patient is provided and may comprise radar means for generating at least one radio frequency, at least one antenna configured for positioning adjacent the skin of the patient, the at least one antenna is additionally configured to at least one of emit the at least one radio frequency into tissue of the patient and collect the reflected at least one radio frequency from the tissue, calibration means for associating one or more sensed pressure wave values with intentional induced changes in blood pressure of the patient, and systolic and diastolic blood pressure calculation means configured to estimate systolic and diastolic blood pressure values based on curve fitting to part of the pressure wave.

In some embodiments, a method for calculating blood pressure using radio frequency is provided and may comprise emitting at least one radio frequency into the tissue of a patient through at least one antenna, the antenna configured to be positioned on the skin of the patient adjacent an artery, collecting the at least one radio frequency after being reflected from the tissue, and calculating at least one of the Systolic and Diastolic blood pressure based on the reflected at least one radio frequency.

Some embodiments may include at least one of the following additional features (all of the below may be referred to as “additional features”): calibrating the reflected radio frequencies signal amplitude, where calibrating may comprise calculating a radio frequency signal amplitude to pressure conversion ratio based on the received reflect radio frequency; the ratio is calculated based on the reflected radio frequency signal amplitude when the tissue is at two different elevations; a sensor is associated with the at least one antenna; determining unwanted relative movement of the at least one antenna relative to the tissue of the patient (e.g., based on sensor date); compensating the calculation of the artery diameter measurement based on the determined unwanted relative movement; calculating the difference between the systolic and diastolic pressures by means of difference of the calibrated radio frequencies signal amplitude; optionally calculating the ratio of the systolic and diastolic pressures by means of curve fitting to the pressure wave; the at least one radio frequency is emitted at a repetition rate sufficient to capture changes in the artery diameter throughout the heart pulse cycle; and compensating the reflected at least one radio frequency, where compensating may comprise estimating the impact of the distance of the antenna(s) from the skin variation on the signals' amplitudes, using the amplitude and/or phase of the signal reflected of other tissue layers, and/or the ratio of polynomials of the amplitude and/or phase from various tissue layers of the at least one reflected radio frequency.

In some embodiments, a system for calculating blood pressure using radio frequency is provided and may comprise at least one antenna configured for positioning adjacent the skin of the patient, the at least one antenna is additionally configured to at least one of emit the at least one radio frequency into tissue of the patient and collect the reflected at least one radio frequency from the tissue, radar means for generating the at least one radio frequency, a processor having computer instructions operational thereon to cause the processor to: associate one or more sensed pressure wave values with intentional induced changes in blood pressure of the patient; and calculate the difference between the Systolic and Diastolic blood pressures based on reflection amplitude.

In some system embodiments, the computer instructions may be additionally configured to cause the processor to perform functionality noted in the additional features noted above.

The following paragraphs prior to the Brief Description of the Drawings are incorporated from WO/2013/118121 by Barak.

The RADAR unit may be a Stepped Frequency RADAR or a pulsed RADAR, or may be adapted to use FMCW (Frequency Modulation Continuous Wave) with a sweep time of 10 psec and the sampling frequency of the ADC (Analog to Digital Converter) is 3.2 MHZ. The FMCW RADAR unit may use triangle wave modulation, multirate ramp, triangular wave modulation or wideband sine-wave modulation. The interference may be eliminated using Multiple Reference ANC (Adaptive Noise Cancellation), Recursive Least Squares (RLS), Least Mean Square (LMS), Filtered-X LMS (FxLMS) or FuLMS (Filtered-u LMS). Preferably, the heart-rate sensor may be integrated into a wristwatch or wristband.

The heart-rate sensor may include a voltage controlled oscillator (e.g., a variable frequency ring oscillator, fabricated using standard CMOS (Complementary Metal-Oxide Semiconductor) or BiCMOS (Bipolar CMOS) technologies) modulated by a ramp signal spanning the full signal bandwidth from 3.1 to 10.6 GHz with a typical sweep time of 10 μsec. The VCO (Voltage Controlled Oscillator) output may be coupled to the antenna and to the LO (Local Oscillator) input of a mixer that mixes with the VCO signal to produce an IF (Intermediate Frequency) signal which is filtered by a Low Pass Filter (LPF) and amplified by an IF amplifier, before being sampled by an ADC. The frequency variation of the oscillator may be in discrete steps. The antenna may be a dual planar cross-bow dipole antenna which comprises two orthogonal broadband dipoles, a single arm spiral antenna, a single broadband dipole antenna or a slot antenna. The frequency analysis for splitting the superposition may be performed by using DFT (Discrete Fourier Transform), a chirp-Z transform, or an analog filter bank.

The RADAR unit may operate at a duty cycle below 1%. The FMCW chirp width may be at least 5 GHz. The heart-rate sensor may include circuitry for cancellation of interference caused by a movement of the sensor, by using signals from a plurality of time bins. Preferably, the oscillator bandwidth is more than 5 GHz. The heart-rate sensor may comprise two orthogonal antennas, one for transmitting and one for receiving. The heart-rate sensor may further include a radio transmitter to relay heart rate data to a remote receiver or terminal and a wrist strap enabling wearing the sensor on a wrist.

### BRIEF DESCRIPTION OF THE DRAWINGS

FIG. 1 shows a simplistic example of these tissue layers, for understanding their interaction with radio waves;

FIG. 2 shows the antennae positioning on the subject wrist above the Radial artery according to some embodiments of the disclosure;

FIG. 3 shows the positioning of the dual slot antenna on the subject's wrist according to some embodiments of the disclosure;

FIG. 4 depicts the arterial pressure change versus time, when the measurement is performed on the subject's wrist, and the hand is positioned in the upper and lower positions according to some embodiments of the disclosure;

FIG. 5 depicting the detected signal from the radial artery in the same positions according to some embodiments of the disclosure;

FIG. 6 shows the compensated detected signal approximating a representation of the Pressure Wave described in calibrated pressure units according to some embodiments of the disclosure;

FIG. 7 shows the exponential fitted curve to the tail of the pressure wave according to some embodiments of the disclosure;

FIG. 8 shows a prior art wearable device for measuring arterial blood pressure for, on a wrist of a person, for comparison to a wearable device, on a wrist of person, of the present invention shown in FIG. 9; and

FIG. 9 shows a wearable device for measuring arterial blood pressure of the present invention, on a wrist of a person, illustrating a difference compared to FIG. 8 in how the devices can fit to the body of a wearer of the device.

FIGS. 1001-1007 correspond to FIGS. 1-7 in WO/2013/118121 by Barak. The brief descriptions of FIGS. 1001-1007 and the detailed descriptions of FIGS. 1001-1007 are incorporated from WO/2013/118121 by Barak.

FIG. 1001 is a top level block diagram usable in an embodiment of the invention of PCT/IL2013/050113;

FIG. 1002 is a cross section of a human arm, showing the location of the radial artery;

FIG. 1003 is a simplified block diagram of the sensor integrated into a wristwatch, usable in an embodiment of the invention;

FIG. 1004 is a block diagram of the sensor, usable in an embodiment of the invention;

FIG. 1005 is a block diagram of an alternative embodiment using a single antenna;

FIG. 1006 shows the waveform of the detected pulse signal, and the points of extraction of heart-rate related measurements;

FIG. 1007 shows a dual planar cross bow dipole antenna, used by the present invention; and

FIG. 1008 shows an expanded view of the central region of FIG. 1007.

### DETAILED DESCRIPTION OF EMBODIMENTS

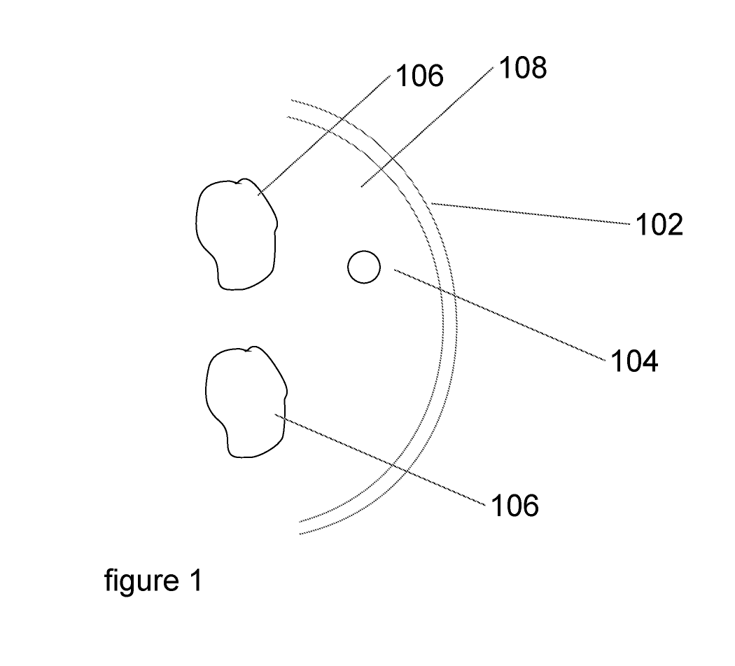

In some embodiments, an ultrawideband (UWB) microwave signal is radiated into the body tissue, preferably in a body location where an artery is close to the skin. This position may be on the wrist, above the Radial artery. In some embodiments, the reflected signal is the complex summation of multiple reflections, each reflection representing the signal reflected from successively ascending depth into the body tissue caused by the complex dielectric constant change in the different tissue layer boundaries. A simplistic example of these tissue layers is described in FIG. 1, which represents a cross section of a subject's arm close to the wrist, which is a preferred location, according to some embodiments, to attach the apparatus/system/device. As shown, 102 represents the skin layer, 104 represents the Radial Artery, 106 represents the muscle tissue and 108 represents the bones. The amplitude of each reflection, referred to hereafter as Sₜ, t being the specific tissue causing the reflection, represents the radar-cross-section (ReS) of the associated tissue layer. For example, S_{artery} is the time varying amplitude of the signal reflected of the muscle-artery boundary.

In some embodiments, for proper separation of the reflected signal off the artery, from the signal reflected from other tissue elements, the signal bandwidth is preferably as high as possible, and preferably, at least more than 2 GHz (e.g., between about 2 GHz and about 11 GHz, and in some embodiments, between about 3.1 GHz to about 10.6 GHz).

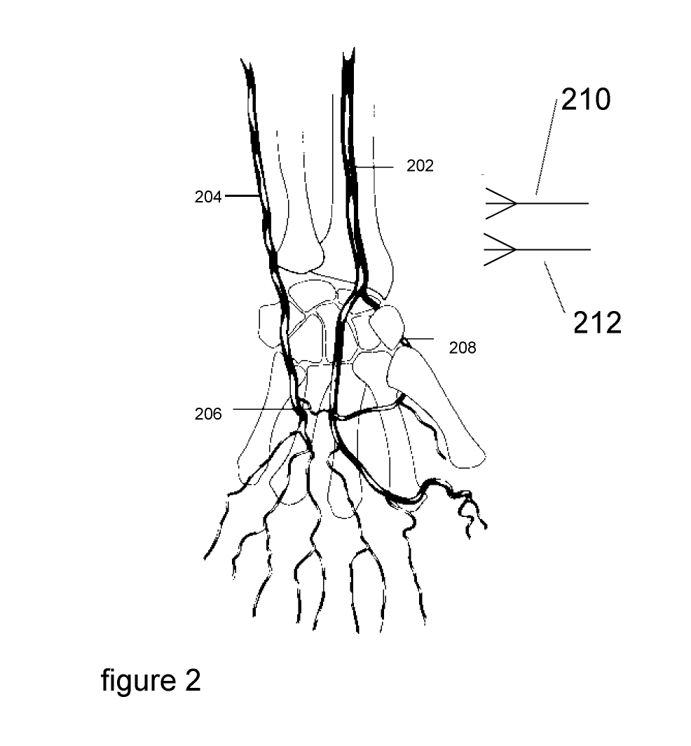

In some embodiments, transmit and receive antennas are provided, and are positioned on the subject wrist above the Radial artery, as shown in FIG. 2. The radiated signal may then be transmitted at a repetition rate sufficient to capture the changes in the artery diameter throughout the heart pulse cycle. This rate is preferably 30 samples per second or higher than 30 samples per second, to properly describe the pressure wave details. The resulting signal associated with the artery, S_{artery}, may correspond to a sampled representation of the artery diameter, and may be essentially repetitive in synchronization with the heart pulsing cycle. This signal may then be referred to as the Pressure Wave.

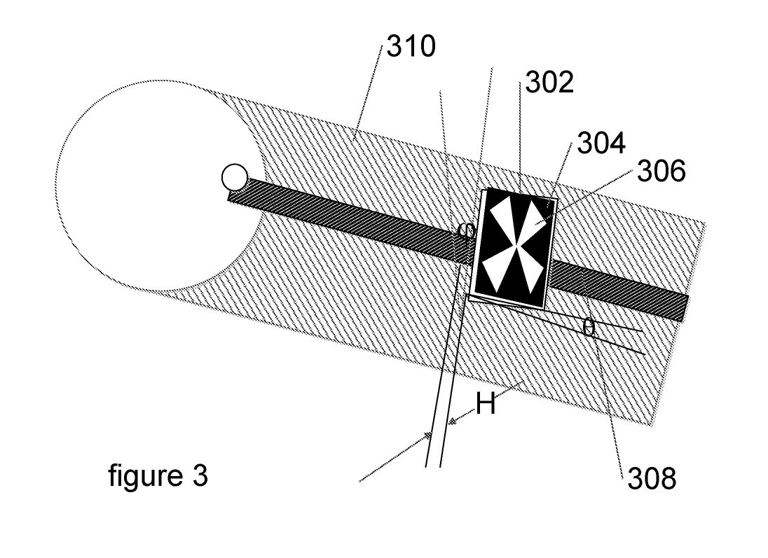

In some embodiments, the transmit and receive antennas are arranged close to the skin surface of the limb for which arterial measurement will be obtained close to the limb skin surface. To prevent direct coupling between the two antennas, the antennas may be positioned orthogonally one to the other. In some embodiments, these antennas may be implemented as, for example, printed slot antennas on a dielectric substrate, as shown in FIG. 3. For illustrative purposes, the limb is described schematically as a cylinder 310. In the limb, the artery 308 is shown inside the limb close to the skin surface. The collective transmit/receive antenna 312 may be positioned essentially tangential to the skin surface, with positioning errors θ and φ representing the rotation angles relative to the skin surface, and H denoting the separation of the antennas for the skin. One and/or another of the antennas top side may be covered with a conductor 304 outlining the slot 306. This slot is the union of the transmit and receive slots.

The exact shape, the faces (to be covered with the metal), and slot are design parameters. In some embodiments, the antenna may include a multiplicity of one or more dielectric layers, with metal conductors which may be located on at least some of the interfaces. For example, positioning the metal/slot layer on the inner side of the dielectric slab, and covering the backside with a continuous metal layer.

Accordingly, in some embodiments, the amplitude of the signal associated with artery, relates to this artery section diameter. The artery diameter is related to the arterial pressure.

In such embodiments, to first order, S_{artery}(t)=α*p(t)+K, S_{artery} is this signal strength, p(t) being the time varying artery pressure and a is an unknown calibration constant, and K is a constant associated with the signal reflected from artery in the unrealistic condition where the arterial pressure is zero.

However, in some embodiments, the signal may also be highly dependent on the antenna-to-organ spacing and/or orientation, as denoted by H in FIG. 3. This dimension, as well as the antenna orientation vis-a-vis the limb, may vary during calibration or during measurement, introducing significant measurement errors.

In some embodiments, this error may be compensated by, for example, estimating the impact of the variation of the distance of the antenna or antennas from the skin impact on the signals' amplitude, using the amplitude and/or phase of the signal reflected of other tissue layers, and in some embodiments, mainly the skin layer, which produces the strongest echo. This estimate may then be used to modify the S_{artery} value, so the result is compensated against relative antenna-limb movements, for example.

In some embodiments, this compensation can be implemented as an interpolation of a look up table and the ratio of polynomials of the reflection amplitude and phase from various tissue layers.

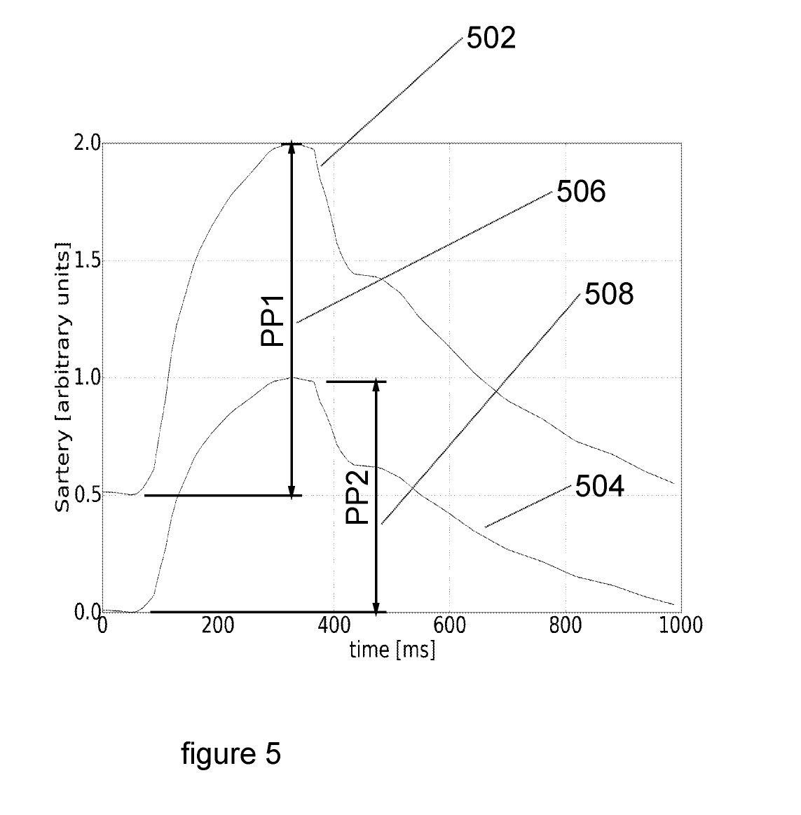

In some embodiments, compensation may rely on the proposition that the Pressure Wave peak-peak magnitude is invariant to limb position. Pressure Wave peak-peak magnitude means the actual pressure difference between the diastolic pressure and the systolic pressure in the artery. This difference in pressures can either be assumed to not vary as a function of limb position or to vary based upon a specified artery diameter/pressure nonlinear relationship. The detected S_{artery} may vary between the calibration measurements because of shift of the sensor location relative to the artery. Thus, the peak-peak difference can be used to compensate for changes in S_{artery} measurements due to shift of sensor position between measurements use for calibration. This scenario is illustrated in FIG. 5, which depicts the detected S_{artery} signals Sₐ₁, Sₐ₂ defined as 502, 504 at the subject's arm positions in a down position and an up position, respectively. The Peak-Peak measurement 506, 508 of these signals may be defined as PP1 and PP2 respectively.

To that end, and in accordance with some embodiments, the Calibration of Sₐ₂ follows the following procedure: (1) let PP1=max(Sₐ₁)−min(Sₐ₁); (2) let PP2=max(Sₐ₂)−min(Sₐ₂); and (3) Sₐ₂comp=Sₐ₂*PP1/PP2.

In a similar manner, compensated S_{artery} signals, in some embodiments, can be achieved at various other heights.

Any of these calibration cases result in compensated signals representing the Pressure Wave in the artery, with an unknown calibration constant u. This calibration constant can be found, for example, by measuring S_{artery} at a plurality of different arterial pressures, where the difference in pressure is known, relating only to the hydrostatic pressure difference (for example). In some embodiments, the pressure difference is created by the subject hand being raised, such that the wrist is lifted by a known height.

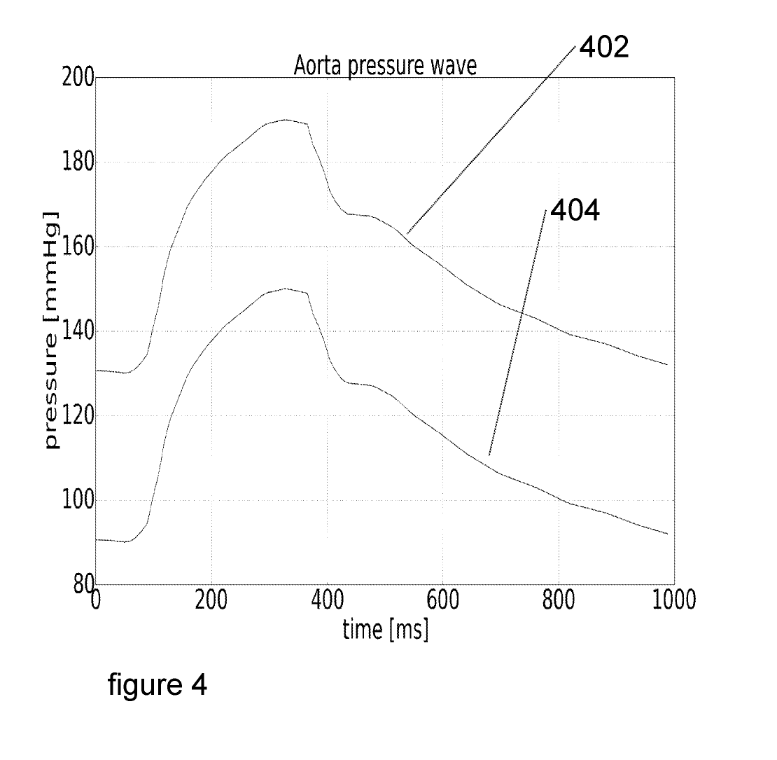

FIG. 4 depicts the arterial pressure change versus time, according to some embodiments, whereby traces 402 and 404 represent the arterial pressure in the subjects arm in the lower position, and in the upper position, respectively. The difference of the mean of the two Pressure Waves, at lower and higher positions of the wrist, referred to hereafter as ΔS, is used for this calibration. The shift in height creates a shift in the arterial pressure wave by the quantity ΔP=ρ*g*ΔH, ρ being the blood specific gravity, g being the gravitational acceleration constant.

Accordingly, when the subject lifts his hand from the vertical downwards position to the vertical upwards position, and the height difference is known or can be assumed knowing the subject height and gender. For example, in humans, there exists a practically fixed proportion between the body height and limb lengths. Thus, a processor/controller may be provided which may be programmed to receive data representing a subject's height, gender, and other physiological data, to calculate the distance. This data may be referred to as the Subject Physiological Data.

In some embodiments, the height difference can be estimated by including an accelerometer or gyro on the limb (e.g., the accelerometer being integrated as part of one and/or another of the radar antennas, or other structure which is mounted to the limb, e.g., a housing and/or frame, hereinafter referred to as “the housing”), and the vertical acceleration may be integrated into the algorithm for determining the vertical distance in the processor.

In some embodiments, the vertical distance/height can be approximated by using an optical camera embedded in the housing that, by using the Subject Physiological Data, can be used to estimate the vertical shift/distance. For example, a processor can be configured to process image data for estimating movement, by, for example, estimating the orientation of the subjects body (e.g., horizontal, vertical), using recognizable object(s) in an image taken at different times (e.g., lights, doors, floor, windows, and the like), and/or optically estimating the hand movement compared to the known subject's body length.

Accordingly, in some embodiments, the time average difference in Pressure Wave, together with the estimated height difference ΔH, the known acceleration constant, the assumed constant blood specific gravity, allow the extraction of the parameter α. α=ΔS/ΔP.

In some embodiments, in a more precise calibration, the value α can be assumed to be a function of the Pressure Wave, and so, calibration is taken at a plurality of elevation positions of the arm/limb to approximate the nonlinear characteristic of S_{artery} versus the arterial pressure. This results in a distinct injective mapping of signal S_{artery} with the blood pressure.

In some embodiments, calibration may utilize other acceleration sources in addition to gravity (see paragraph [0034] above). For example, acceleration of the arm/limb by the subject by deliberate movement, this acceleration can be measured by an accelerometer (provided for in the housing, for example), and the resulting change in S_{artery} can be correlated to the measure acceleration to extract the calibration constant α.

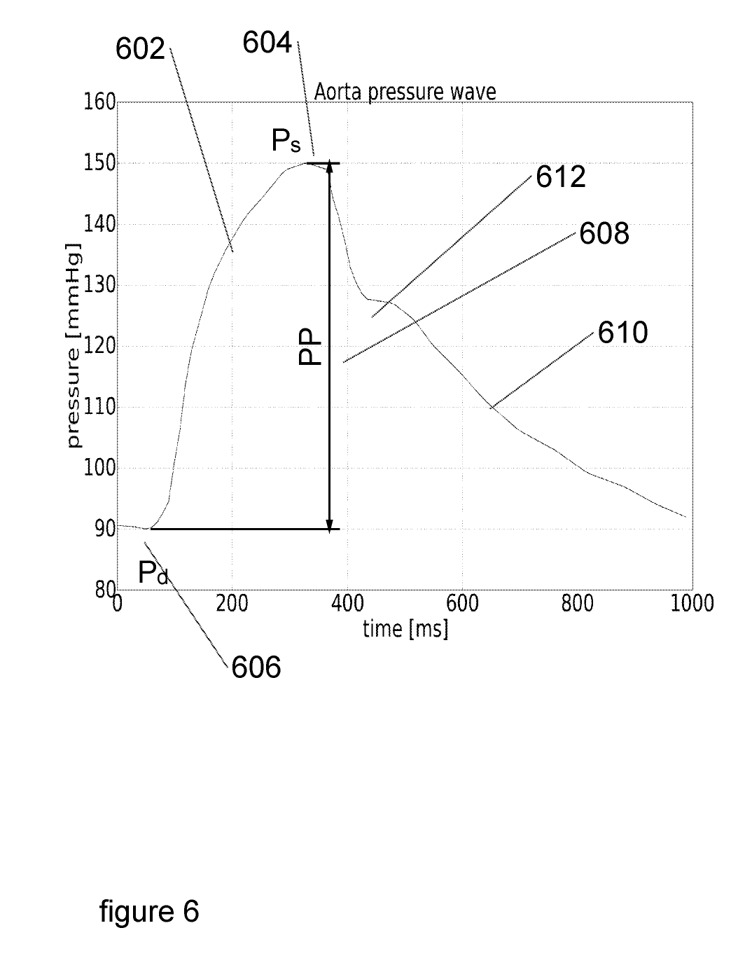

FIG. 6 shows a compensated S_{artery} signal 602 approximating a representation of the Pressure Wave, described in calibrated pressure units. As shown, the wave has a distinct peak 604 and distinct valley 606 representing the Systolic and Diastolic pressures Pₛ and P_{d}, respectively. The pressure difference between the latters is defined as PP 608. PP is a precise measurement. However, Pₛ and P_{d} are not estimated without the unknown K. The shape of the pressure tail 610 following the dicrotic notch 612, approximately follows an exponential curve (e.g., as proposed by Otto Frank). This function shape is understood to correlate to the pressure rate of change being linearly related to the pressure difference between the artery pressure and the vein pressure. The vein pressure is usually between 10 mm Hg and 20 mm Hg, and is assumed a constant in some embodiments of the disclosure.

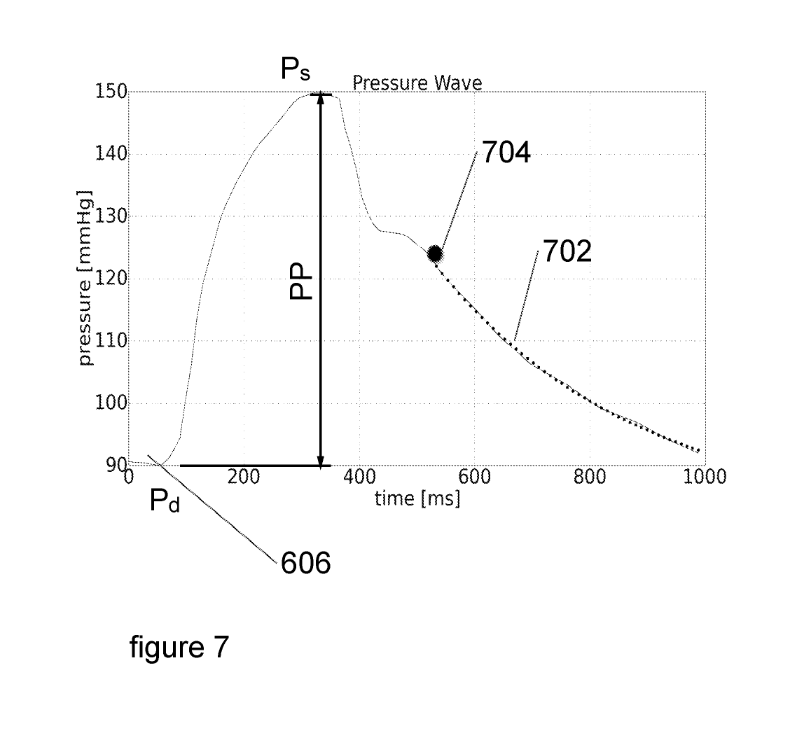

In some embodiments, an exponential function P=P0+P1*e^{-P2(t-t0)} is matched in sense of “minimum norm 2 error” to the tail 610. P0 is some constant pressure for example the vein pressure. For example, FIG. 7 shows the resulting fit 702. Using an arbitrary point 704 on the fitted curve, and the diastolic pressure 606 enables the solution of two simultaneous equations, the known difference equation D=Value(704)−Value (606) and the ratio Deriv(704)/Deriv(606) which must have the same value, are sufficient to solve for P1 and P2 and calculate the absolute values Pₛ and P_{d}.

In some embodiments, a matching function may be an exponential decaying sine wave function representing the non-uniform frequency characteristic of the artery tree. Matching at additional point will enable the extraction of the function parameters, and estimate the absolute values of Pₛ and P_{d}. In this case a mathematical manipulation of the decaying oscillation is needed to separate the exponent, whose derivatives are necessary for calculation of the absolute Systolic and Diastolic pressures, from the complex shape. In some embodiments, this is done by curve fitting to a numerical model representing the artery tree wave reflections as described in “Arterial blood pressure measurement and pulse wave analysis-their role in enhancing cardiovascular assessment” by Alberto P Avolio et al, doi: 10.1088/0967-3334/31111ROI.

In some embodiments, it may be beneficial to match the exponential decaying sine wave to the Aortal pressure, as approximated using the generalized transfer function as described in “Pulse wave analysis” by Michael F. O'Rourke et al., J Hypertens Suppl. 1996 December; 14(5):SI47-S7. In some embodiments, the calibrated pressure wave may be translated to the pressure as would be measured in the brachial artery, and the central Aorta blood pressure, using a model of the artery tree. Preferably this model is a spectral model. Time domain model is mathematically equivalent, and can also be used.

Communication between various components, including a processor which includes computer instructions operable thereon which are configured to at least one of control the disclosed devices and systems, and calculate diastolic and systolic values, as well as calibration of values, can be wired communication, and/or wireless via an analog short range communication mode, or a digital communication mode including, for example, WI-FI or BLUETOOTH®. Additional examples of such communication can include communication across a network. Such a network can include a local area network (“LAN”), a wide area network (“WAN”), or a global network, for example. The network can be part of, and/or can include any suitable networking system, such as the Internet, for example, and/or an intranet.

Generally, the term “Internet” may refer to the worldwide collection of networks, gateways, routers, and computers that use Transmission Control Protocol/Internet Protocol (“TCP/IP”) and/or other packet based protocols to communicate there between.

In some embodiments, the disclosed systems and devices may comprise one or more transmission elements for communication between components thereof. In some embodiments, the transmission element can include at least one of the following: a wireless transponder, or a radio-frequency identification (“RFID”) device. The transmission element can include at least one of the following, for example: a transmitter, a transponder, an antenna, a transducer, and/or an RLC circuit or any suitable components for detecting, processing, storing and/or transmitting a signal, such as electrical circuitry, an analog-to digital (“A/D”) converter, and/or an electrical circuit for analog or digital short range communication.

In some embodiments, a controller/processor according to some embodiments and/or any other relevant component of disclosed devices and systems can include a memory, a storage device, and an input/output device. Various implementations of some of embodiments disclosed, in particular at least some of the processes discussed (or portions thereof), may be realized in digital electronic circuitry, integrated circuitry, specially configured ASICs (application specific integrated circuits), computer hardware, firmware, software, and/or combinations thereof (e.g., the disclosed processor/controllers). These various implementations, such as associated with the disclosed devices/systems and the components thereof, for example, may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs, software, software applications or code) include machine instructions/code for a programmable processor, for example, and may be implemented in a high-level procedural and/or object-oriented programming language, and/or in assembly/machine language. As used herein, the term “machine-readable medium” refers to any computer program product, apparatus and/or device (e.g., nontransitory mediums including, for example, magnetic discs, optical disks, flash memory, Programmable Logic Devices (PLDs)) used to provide machine instructions and/or data to a programmable controller/processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computing device which includes a display device (e.g., a LCD (liquid crystal display) monitor and the like) for displaying information to the user and a keyboard and/or a pointing device (e.g., a mouse or a trackball, touchscreen) by which the user may provide input to the computer. For example, this program can be stored, executed and operated by the dispensing unit, remote control, PC, laptop, smart phone, media player or personal data assistant (“PDA”). Other kinds of devices may be used to provide for interaction with a user as well.

For example, feedback provided to the user may be any form of sensory feedback (e.g., visual feedback, auditory feedback, or tactile feedback), and input from the user may be received in any form, including acoustic, speech, or tactile input. Certain embodiments of the subject matter described herein may be implemented on a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components.

Any and all references to publications or other documents, including but not limited to, patents, patent applications, articles, Web pages, books, etc., presented anywhere in the present application, are herein incorporated by reference in their entirety. Example embodiments of the devices, systems and methods have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims directed to one and/or another embodiment of one and/or another invention, which are supported by the present disclosure and their equivalents. Moreover, embodiments of the subject disclosure may include methods, systems and devices which may further include any and all elements/features from any other disclosed methods, systems, and devices, including any and all features corresponding to blood pressure measurement. In other words, features from one and/or another disclosed embodiment may be interchangeable with features from other disclosed embodiments, which, in turn, correspond to yet other embodiments. Furthermore, one or more features/elements of disclosed embodiments may be removed and still result in patentable subject matter (and thus, resulting in yet more embodiments of the subject disclosure). Still further, some embodiments are distinguishable from the prior art due to such embodiments specifically lacking one or more features which are found in the prior art. In other words, some embodiments of the disclosure include one or more negative limitations to specifically note that the claimed embodiment lacks at least one structure, element, and/or feature that is disclosed in the prior art.

The following aspects of the invention appeared as claims in the U.S. provisional application No. 62/024,403.

One aspect of the invention is (1) a blood pressure calculation apparatus configured to calculate blood pressure of a patient based on sensing an artery pressure wave of the patient, comprising: radar means for generating at least one radio frequency; at least one antenna configured for positioning adjacent the skin of the patient, the at least one antenna is additionally configured to at least one of emit the at least one radio frequency into tissue of the patient and collect the reflected at least one radio frequency from the tissue; calibration means for associating one or more sensed pressure wave values with intentional induced changes in blood pressure of the patient; and Calculation means to calculate the difference between the Systolic and Diastolic blood pressures, configured to estimate systolic and diastolic blood pressure values difference based on reflection amplitude. Dependent aspects are (2) the apparatus further including Systolic and Diastolic blood pressure calculation means to calculate, configured to estimate systolic and diastolic blood pressure values based on curve fitting to part of the pressure wave; (3) the apparatus where the calibration means calibrates the calculated systolic and diastolic values based on data collected from the arm of the patient corresponding to at least one of raising or lowering of the arm; (40 the apparatus wherein the calculation means determines the systolic and diastolic blood pressure based on the reflected radio frequency; (5) the apparatus wherein the calculation means determines the diameter of an artery adjacent the skin of the patient based on the reflected radio frequency; (6) the apparatus wherein the radar means generates a multiplicity of radio frequencies, the difference between the highest and lowest frequency at least 2 GHz; (7) the apparatus wherein the at least one radio frequency comprises a plurality of radio frequencies.

One aspect of the invention is (8) a method for calculating blood pressure using radio frequency comprising: emitting at least one radio frequency into the tissue of a patient through at least one antenna, the antenna configured to be positioned on the skin of the patient adjacent an artery; collecting the at least one radio frequency after being reflected from the tissue; and calculating at least one of the Systolic and Diastolic blood pressure based on the reflected at least one radio frequency. Dependent aspects are (9) the method wherein calculating includes calibrating the reflected radio frequencies; (10) the method wherein calibrating comprises calculating a radio frequency signal to pressure conversion ratio based on the received reflect radio frequency; (11) the method wherein the ratio is calculated based on the reflected radio frequency when the tissue is at two different elevations; (12) the method wherein a sensor is associated with the at least one antenna, and wherein the method further comprises determining unwanted relative movement of the at least one antenna relative to the tissue of the patient; (13) the method further comprising compensating the calculation of the artery diameter measurement based on the determined unwanted relative movement; (14) the method wherein calculating the systolic and diastolic pressure includes approximating a time derivative of the blood pressure; (15) the method wherein the at least one radio frequency is emitted at a repetition rate sufficient to capture changes in the artery diameter throughout the heart pulse cycle; (16) the method further comprising compensating the reflected at least one radio frequency; (17) the method of claim wherein compensating comprises estimating the impact of distance of the antenna(s) from the skin on the amplitude of the signals, using the amplitude and/or phase of the signal reflected of other tissue layers; (18) the method wherein compensating comprises an interpolation of a look up table and the ratio of polynomials of the amplitude and/or phase from various tissue layers of the at least one reflected radio frequency; (19) the method wherein the at least one radio frequency comprises a plurality of radio frequencies.

One aspect of the invention is (20) a system for calculating blood pressure using radio frequency comprising: at least one antenna configured for positioning adjacent the skin of the patient, the at least one antenna is additionally configured to at least one of emit the at least one radio frequency into tissue of the patient and collect the reflected at least one radio frequency from the tissue; radar means for generating the at least one radio frequency; a processor having computer instructions operational thereon to cause the processor to: associate one or more sensed pressure wave values with intentional induced changes in blood pressure of the patient; and calculate the difference between the Systolic and Diastolic blood pressures based on reflection amplitude. Dependent aspects are (21) the system wherein the computer instructions are additionally configured to cause the processor to calibrate the reflected radio frequencies; (22) the system wherein the computer instructions are additionally configured to cause the processor to calculate a radio frequency signal to pressure conversion ratio based on the received reflect radio frequency; (23) the system wherein the ratio is calculated based on the reflected radio frequency when the tissue is at two different elevations; (24) the system further comprising a sensor configured to be associated with the at least one antenna, and wherein the computer instructions are additionally configured to cause the processor to determine unwanted relative movement of the at least one antenna relative to the tissue of the patient; (25) the system wherein the computer instructions are additionally configured to cause the processor to compensate the calculation of the artery diameter measurement based on the determined unwanted relative movement; (26) the system wherein calculating the diastolic pressure includes approximating a time derivative of the blood pressure; (27) the system of wherein the at least one radio frequency is emitted at a repetition rate sufficient to capture changes in the artery diameter throughout the heart pulse cycle; (28) the system wherein the computer instructions are additionally configured to cause the processor to compensate the reflected at least one radio frequency; (29) the system wherein compensating comprises estimating the impact of the variation of distance of the antenna(s) from the skin on the signals' amplitudes, using the amplitude and/or phase of the signal reflected of other tissue layers; (30) the system wherein compensating comprises an interpolation of a look up table and the ratio of polynomials of the amplitude and/or phase from various tissue layers of the at least one reflected radio frequency; (31) the system wherein the at least one radio frequency comprises a plurality of radio frequencies.

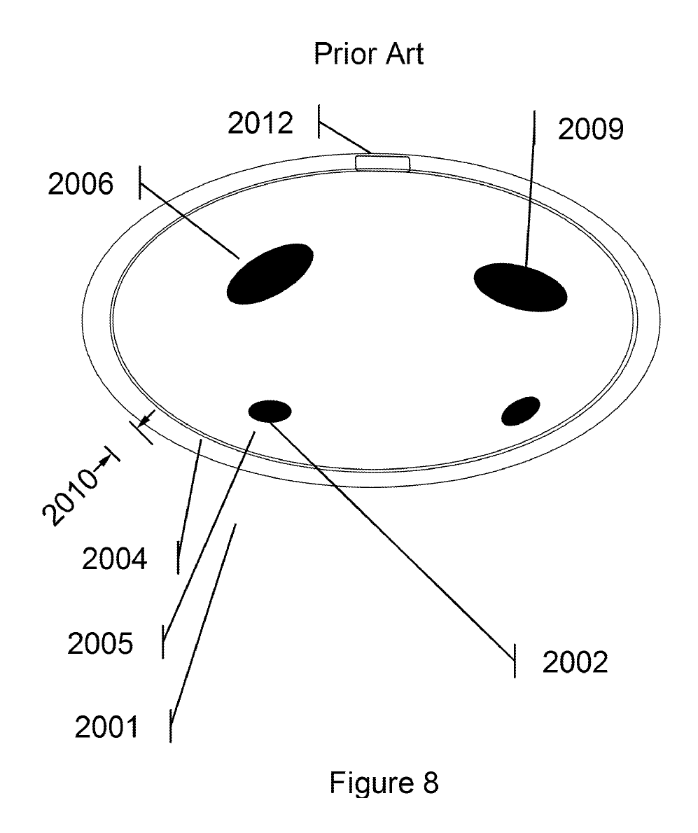

FIG. 8 shows a prior art wearable device on a wrist of a person, including wristband 2001 in which a prior art PPG sensor 2012 is embedded. Inside the cross-section 2004 of the wrist (unnumbered) there are bones 2006; 2009; and radial artery 2002. 2011 represents the distance between the wrist strap and the wrist, and 2010 represents the thickness of the wrist strap. The wrist strap as shown is substantially thicker than the distance between the wrist strap and the wrist. In operation, the wrist strap must maintain the PPG sensor in contact with the exterior surface of the wrist, which means that the distance 2011 between the wristband and the wrist must be essentially non-existent (zero) around the wrist so that the PPG sensor is maintained in contact with the wrist. That requires a tight fitting wristband. This requirement for a tight fitting wristband is disadvantageous.

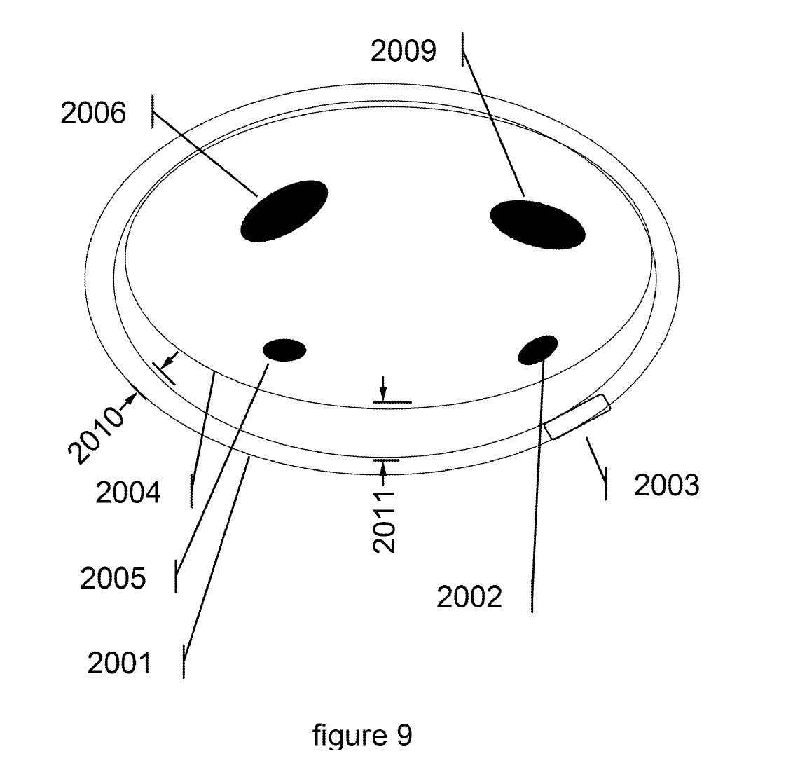

FIG. 9 shows a wearable device for measuring arterial blood pressure of the present invention, on a wrist of a person. FIG. 9 shows the wearable device including wristband 2001 in which EM sensor 2003 is embedded (or otherwise mechanically attached). 2004 represents the cross-section of a wrist or a person. 2006; 2009 represent bones in the wrist. 2005 represents the ulnar artery in the wrist. 2011 represents the shortest distance between the surface of the wrist and one point along the wristband 2001. 2010 represents the thickness of the wrist band (in a cross-section perpendicular to the extension of the limb encircled by the wristband). FIG. 9 shows sensor 2003 separated from the wrist by a distance and therefore not in contact with the wrist. As shown, the distance between sensor 2003 and the surface of the wrist is greater than the thickness of the wristband 2001. FIG. 9 shows that the distance of the sensor from the wrist, and therefore also from an artery in the wrist need not be rigidly fixed and the sensor 2003 need not be in contact with the wrist, for the sensor to function to provide a signal from which blood pressure and artery pressure can be determined. The removal of the requirement (relative to a PPG sensor) of the sensor being in contact with the skin allows for the sensor of the present invention to be retained relative to the body of a wearer in novel ways, including by a clip to clothing; a lose fitting band around some part or the body; and integrated into some other piece of wearable clothing.

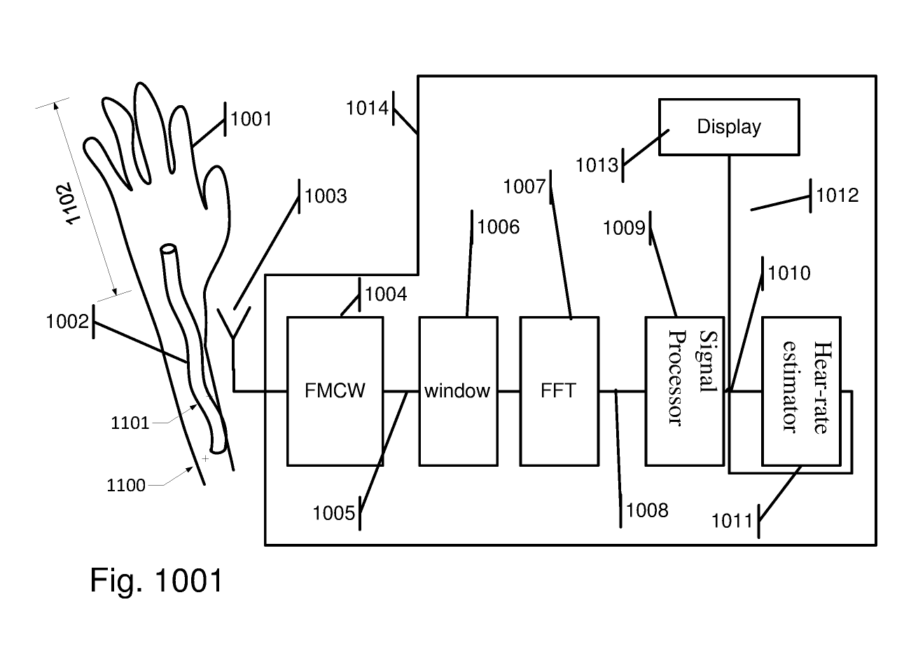

FIG. 1001 shows a simplified block diagram of the sensor proposed by the present invention. The Sensor 1014 is connected to antenna 1003 for sensing the instantaneous volume of blood in the artery 1002 to be measured. A Frequency Modulated Continuous Wave (FMCW) RADAR 1004 transmits microwave signals into the subject limb 1, in this case into the arm, via antenna 1003. The limb represents to the RADAR a multiplicity of tissue targets, each of which at a different distance from the antenna 1003. The RADAR output 1005 includes a superposition of signals, each of which corresponding to a specific tissue target. The frequency of each such a signal is related to the distance of the target, and its amplitude is related to the target's reflection strength, usually referred to as Radar Cross Section (RCS). An FFT function processor 1007, followed by window function circuitry 1006, splits the superposition of target information in output 1005 according to its relative frequency, hence its distance, into a multiplicity of bins (bars that contain energy from a frequency range). Each bin output amplitude represents the RCS of the target at a specific distance from the antenna, which is equivalent to a specific depth inside the limb. Window function 1006 is needed to suppress spectral sidebands originating from the abrupt start and stop of signal 1005 (i.e., from the subsequent processor operating on time truncated data), due to using the FMCW radar.

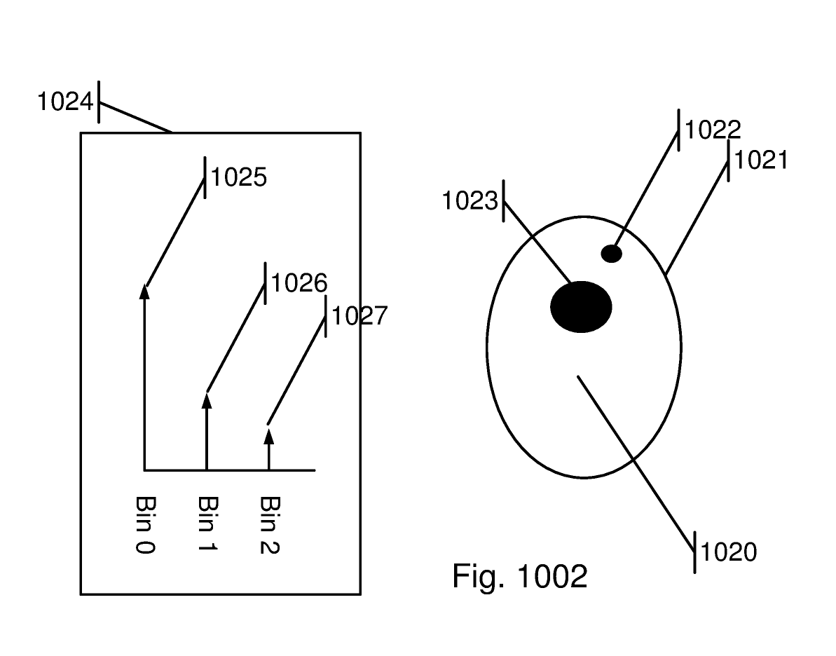

FIG. 1002 shows an example of the FFT (Fast Fourier Transform) output in relation to the limb tissues. In this example, the limb is a human wrist. Its cross section 1020 is shown, and includes for this simplistic illustration, three tissue elements: the skin 1021, the artery 1022, and a bone 1023. The corresponding output of three FFT bins is shown in 1024, also correspond to output signal 105 in FIG. 1001. Bin 0 signal is represented by vector 1025. It is a result of the lowest frequency component of signal 1005, and is related to the nearest tissue, the skin 1021. Bin 1 signal is represented by vector 1026, and is the result of the reflection from the farther situated artery 1022. Bin 2 signal is represented by vector 1027, and is the result of the reflection of the even farther situated bone 1023. The different FFT bins are referred hereafter as range gates, as they represent signals originating from targets in different ranges.

In FIG. 1001, the FFT bins are connected via bus 1008 to signal processor 1009. Signal processor's 1009 task is to filter out the effect of the sensor movement in respect to the limb. Signal processor 1009 generates a signal 1010 that essentially represents only the reflection from the artery. Signal 1010 amplitude is proportional to the artery dilatation, which varies in accordance with the blood pulsating in the artery and therefore, is an essentially periodic signal, whose frequency represents the heartrate. Heart-rate Estimator 1011 measures this frequency and forwards it for display 1013 via signal 1012. In this example, the signal in bin 1 of the FFT represents the dilatation of the artery, and does not include the interfering signals from the other tissue elements, thus eliminating the additive interference described above. The signal in bin 1 does, however, include the multiplicative interference as described above. The signals in the other bins also include this same multiplicative interference, but do not include the time varying component associated with the heart-rate, as they are reflected from other tissue elements. The sensor proposed by for heart rate monitoring detects the multiplicative interference from the other bins, and uses it to cancel the interference on the bin representing the artery dilatation, namely bin 1.

A simple implementation of this cancellation is achieved by dividing the amplitude of the signal resulting from the artery by the amplitude of a signal that does not originate from the artery. Different tissues in a human wrist are located in tight proximity. For example, the distance of the artery from the skin and the artery's depth, is approximately 3.5 mm. In order to separate the signals reflected from so close objects, a large signal bandwidth is needed. For an FMCW application, the signal bandwidth should be at least 3 GHz, and optimal performance can be achieved with a bandwidth of 6 GHz or more. Preferably, the system uses Ultra Wideband (UWB) spectral allocation between 3.1 GHz to 10.6 GHz. By using this frequency range for measuring tissues inside a limb, a range resolution of approximately 3 mm can be obtained. In a preferred embodiment of this invention, the FMCW sweep time is 10 μsec and the sampling frequency of the ADC is set to 3.2 MHZ. With these parameters, the FFT will have 32 bins, with no zero padding (appending one or more zeros to the end of a signal). The FFT bin 0 will represent the reflection from the skin, and the bin 1 will predominantly represent the reflection from the artery. In this preferred setup, the error free signal representing the reflection from the artery can be generated by calculating the weighted ratio of two polynomials, so that the error free resulting signal is calculated by: Sig={b₀+Σ(pᵢ(xᵢ))}/{a₀+Σ(qᵢ(xᵢ))} where pᵢ and qᵢ are polynomials of arbitrary degree and xᵢ are the signal amplitudes corresponding to the various FFT bins. The index I represents the bin number, where i=0 represents bin 0. This calculation is repeated in relation to the FMCW chirp repetition.

The pᵢ and qᵢ coefficients can be fixed values, as in this preferred embodiment. In other embodiments they can be dynamically set by the processor 1009 during a user initiated calibration phase, at start-up, or during the operation of the sensor. This way, different artery depths in different subjects can be handled. These weighting constants can also be adapted to handle the changing dielectric parameters of the subject, caused by physiological changes while exercising or by other reasons. Such physiological changes may be, for example, temperature changes of the tissue, changes in the sweat level on the skin surface, or changing in blood flow. Interference associated with the relative movement, as well as the artifact interference can be eliminated using Multiple Reference ANC, as described in the thesis of “Multiple Reference Active Noise Control” by Yifeng Tu, Virginia Polytechnic Institute and State University March, 1997, the content of which is incorporated herein by reference. The inputs to this noise cancellation algorithm are a multiplicity of FFT bins, and possibly also inputs from an accelerometer or acceleration sensitive device, sensing the acceleration along one or more axes. The adaptive algorithm may include Recursive Least Squares (RLS), least mean square (LMS) and their derivatives, such as Filtered-X LMS (FxLMS) or FuLMS.

The RADAR unit can use the pulsed RADAR method and may use other frequency bands. The bandwidth needed for other RADAR types, for example pulsed RADAR, is at least the same bandwidth needed for the FMCW RADAR. Other types of FMCW RADARs, may be used, including Stepped Frequency Radar (SFR-a radar in which the echoes of stepped frequency pulses are synthesized in the frequency domain to obtain wider signal bandwidth, to achieve high range resolution, without increasing system complexity), triangle wave modulation, multirate ramp, and triangular wave modulation. Wide band sine wave modulation may be used.

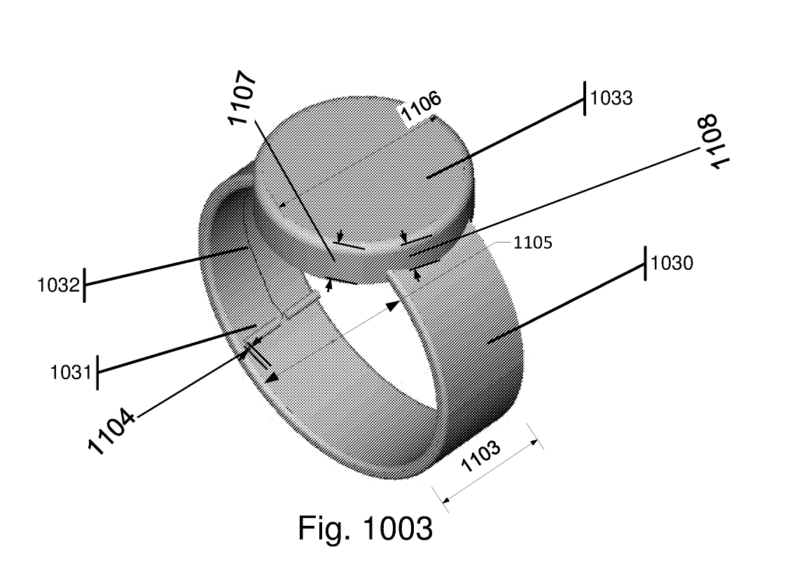

FIG. 1003 shows a preferred embodiment, comprising a housing 1033 and wristband 1030 designed to fit around the wrist of a person. Housing 1033 contains a sensor 1014. Wristband 1030 mechanically connects to housing 1033.

Antenna 1104 resides on or embedded in an inner surface of wristband 1030. Antenna 1104 is coupled to FMCW circuit of sensor 1014 by transmission line 1032. Transmission line 1032 resides on or is embedded in the inner surface of wristband 1030. Transmission line 1032 preferably extends along a midsection of the inner surface of wristband 1030 so that it is equidistant from each lateral edge of the inner surface of wristband 1030. Antenna 1104 preferable extends from one end of transmission line 1032 in both lateral directions toward each lateral edge of wristband 1030. Preferably, antenna 1031 terminates a distance 1104 from each later edge of wristband 1030.

Housing 1033 may (as shown) project up from the outer surface of wristband 1030 by distance 1108 to allow room for sensor 1014. and have a total thickness 1107 in a direction extending away from the center of wristband 1030 that is as larger by a length 1108 than the thickness of wristband 1030 in that same direction. Housing 1033 may extend a distance 1106 in lateral dimensions wherein distance 1106 of the housing extension is greater than the distance 1103 that the wristband 1030 extends in lateral dimensions.

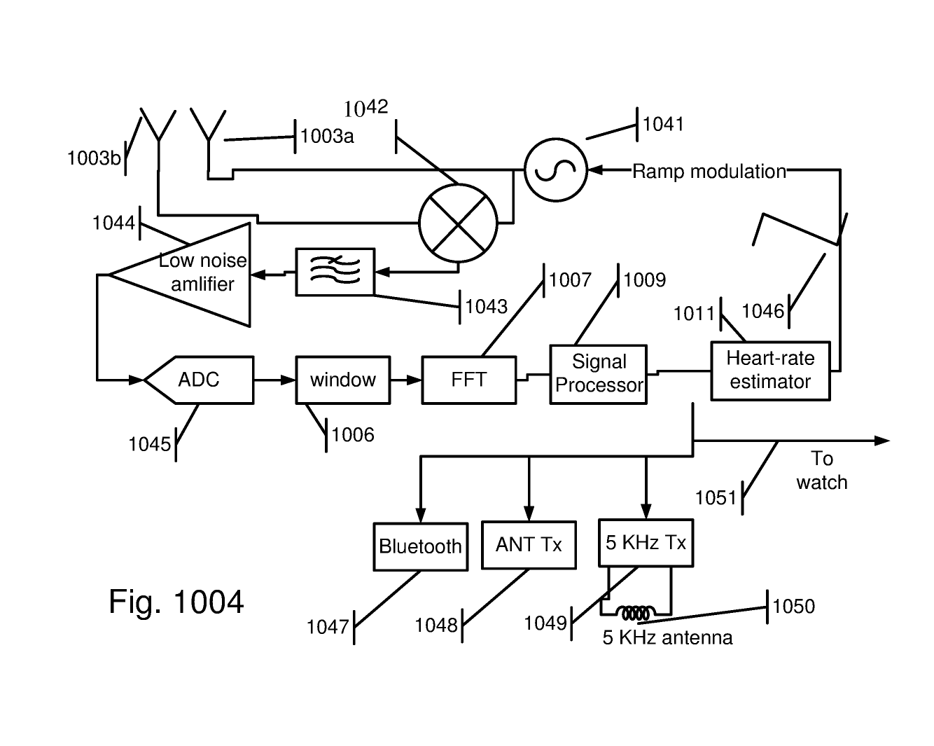

FIG. 1004 is a detailed block diagram of the sensor 1014 to be embedded in the watch, according to a preferred embodiment of this invention. A voltage controlled oscillator (VCO) 1041 (for generating the microwave signal) is modulated by a ramp signal 1046 and spans the full signal bandwidth, which preferably spans from 3.1 to 10.6 GHz. A typical sweep time would be 10 μsec. The selection of this sweep time will cause the detected signal representing the artery to be at approximately 125 KHz. This frequency is high enough to minimize the effect of the semiconductor's shot noise on the Signal to Noise Ratio (SNR). Other sweep times can be selected as needed in different practical implementations. In the preferred embodiment, the VCO output is coupled to the antenna 1003a, and also to the LO input of mixer 1042. In the preferred embodiment, the antenna 1003b receives the reflected signal from the artery, that mixes with the VCO signal in Mixer 1042, to produce an IF signal. This IF signal is filtered by a Low Pass Filter (LPF) 1043 and amplified in IF amplifier 1044, before being sampled by the Analog to Digital converter (ADC) 1045. The IF channel illustrated in FIG. 1004 describes a real signal detection.

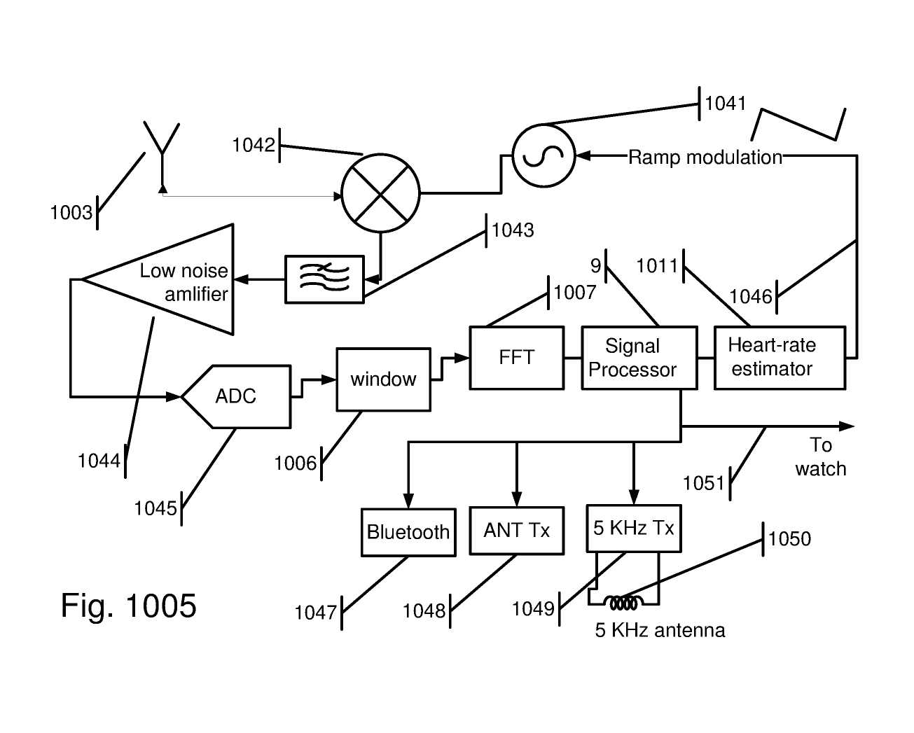

FIG. 1005 shows an alternative embodiment in which dual antenna 1003a and 1003b are replaced by a single antenna 1003. Antenna 1003 is excited using its RF-to-LO parasitic leakage. The mixer 1042 may be purposely designed to leak this signal, which under other circumstances would be unwanted. Alternatively, other coupling mechanisms can be used, including a circulator or a directional coupler.

In both embodiments, the electrical length difference between signal traversing the antenna(s) via the skin reflection and the signal arriving a mixer LO port will define the IF frequency that corresponds to bin 0, or skin reflection. Making this electrical length sufficiently long allows using a single mixer. Complex detection may be used for sufficiently short electrical length difference. Complex detection may be realized by using a quadrature mixer, and a pair each of LPFs, IF amplifiers, and ADCs. For a complex detection, the VCO needs to provide two outputs, with a constant phase difference of 90 degrees between them, which must be frequency independent in the sweep frequency range. The requirement for a large frequency sweep range, and the requirement for a quadrature output, as well as the wish to integrate the microwave circuits and the signal processing circuits into a semiconductor die, can be met by realizing the VCO 1041 as a variable frequency ring oscillator, such as a voltage controlled ring oscillator. Such a quadrature ring oscillator can be fabricated using standard CMOS or BiCMOS technologies.

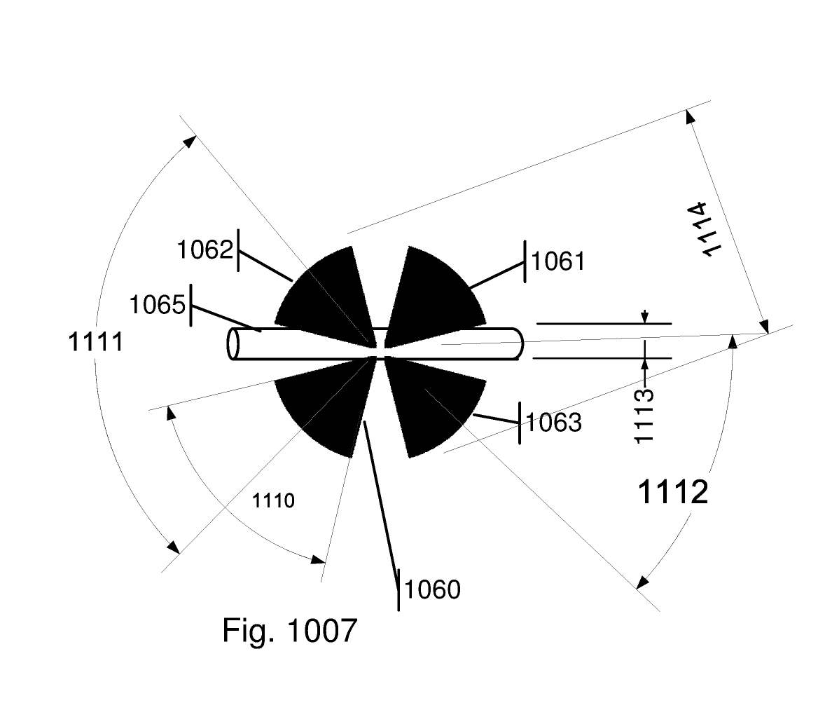

In FIG. 1007 shows a dual planar cross-bow dipole antenna for use in a preferred embodiment in which frequency variation of the oscillator is in discrete steps, as in SFR. Discrete steps allows digital control of the frequency. The antennas 1003a and 1003b are configured to support the broadband signal being used, while minimizing cross-talk. This antenna comprises two orthogonal broadband dipoles, one including conductors 1060 and 1061, and the other including conductors 1062 and 1063. Artery 1065 is located in the X direction, to create an imbalance in the electromagnetic structure and thereby, contributing to the coupling between these dipoles. This allows the diameter or RCS of artery 1065 to generate the received signal in the antenna.

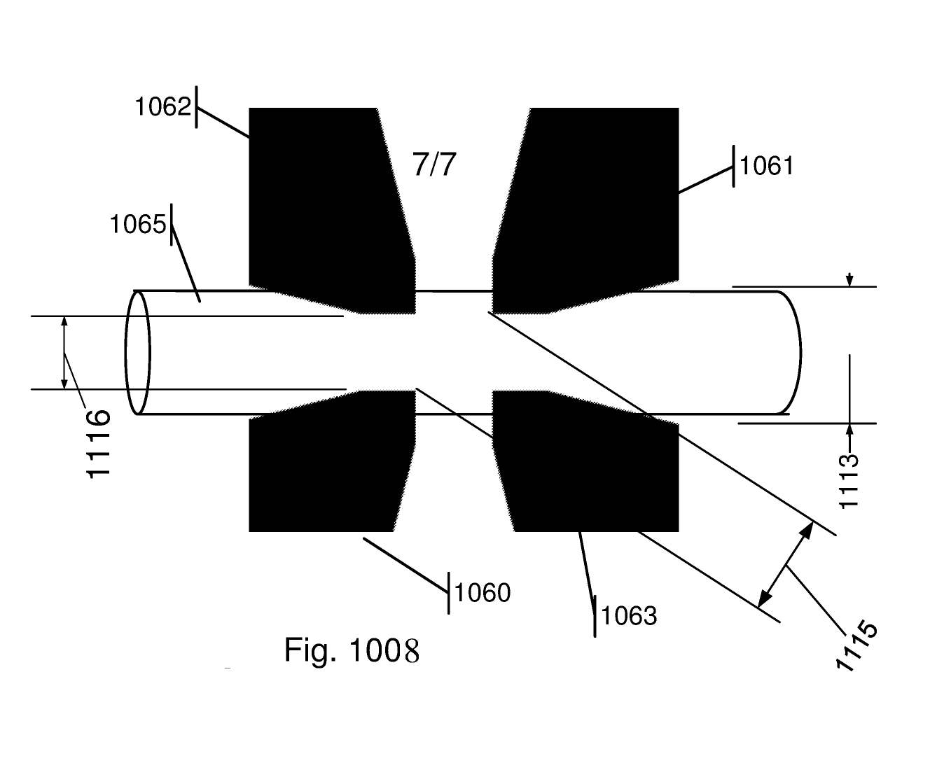

FIG. 1008 shows an expanded view of the central region of FIG. 7 in which the shape and relative locations of elements 1060, 1061, 1062, and 1063 of the antenna, external and internal diameters 1113, 1116, of artery 1065 are more clearly shown. Each element 1060, 1061, 1062, and 1063 is preferably planar and has six straight edges. Outer edges of the element 1060, 1061, 1062, and 1063 are along the perimeter of a square.

In FIG. 1005, single antenna 1003 may be a single arm spiral antenna, a single broadband dipole antenna or a slot antenna. In this case, the reflected signal from the antenna is the received signal.

Embodiments may use other spectral analysis methods, for example including: a DFT, a chirp-Z transform, or an analog filter bank. In a preferred embodiment, a window function 1006 is a Kaiser window with (3=0.5. Other window functions can be used, for example a Tukey Window (tapered cosine) or windows used in connection with Digital Fourier Transforms. In an alternative embodiment, the heart-rate can be estimated using a correlation with a set of predefined wave shapes, each having a slightly different repetition rate. The candidate predefined wave with the highest correlation maximum will be selected as the best estimate. The highest maximum correlation may be detected by using a nonlinear estimator, such as a Maximum Likelihood Sequence Estimator (MLSE).

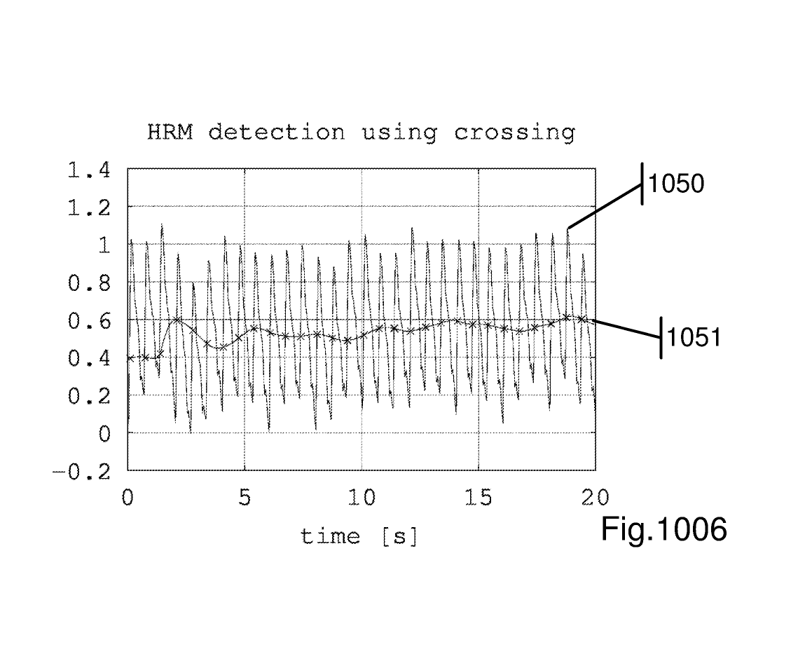

The signal Sig. 1010 resulting from the weighted division shown in FIG. 1004, is of the shape 50 of FIG. 1006. This signal is processed by heart-rate estimator 1011 of FIG. 1004, to produce the estimated heart-rate frequency. The preferred detection method is to compare the signal Sig. of shape 1050 to its running average 1051, and counting the time interval Ti between subsequent positive direction zero crossings, as marked by asterisks on curve 1051. In the preferred embodiment the running average is performed by a fourth order Butterworth filer having a 3 dB bandwidth of 0.5 Hz. The actual heart-rate is calculated by performing a running average on 6 measurements of 60/Ti, where Ti is in seconds. It is possible to use other spectral estimation methods to calculate the heart-rate, for example a Fourier transform. Since the subject heart-rate cannot exceed a few Hertz, the preferred embodiment uses a sampling rate of 10 Hz. The RADAR subsystem needs to active at a duty cycle of 0.01%. This enables the sensor to consume a very low average power, and makes it suitable for coin battery operation. In alternative embodiments, a higher duty cycle can be used to produce a better signal to noise ratio, and to improve the reading accuracy. In this case, multiple measurements can be performed, and the results can be averaged to improve fidelity. In a preferred embodiment, the heart-rate sensor is powered by a CR2032 3V lithium coin battery. It is also possible to aid the powering of the heart-rate sensor with other energy sources, for example a rechargeable battery, a solar cell, or an electric generator that generates electricity from the movement of the subject's hand. Any of these methods of generating and storing electrical energy can be combined. In another embodiment, the heart-rate data can be transmitted to an external recipient that can display the results, such as exercise equipment (e.g., bicycles, exercise treadmills, rowing machines), smart phones, and others. In another embodiment, the sensor may be used to sense the health of a subject, for example a senior person. In this case, the sensor will test the measured heart rate and will compare it to predefined limits or predefined heart rate variation pattern or heart rate variability. If the measurement exceeds predefined limits, it would then communicate this condition via a wireless communication channel, in order, for example, to alert medical care staff.

Many standards for this transmission exist, and a multiplicity of these communication protocols could be supported: 1. The 5 KHz coded protocol 49, which includes a 5 Khz signal that is PPM (Pulse Position Modulation) modulated by a pulse triplet, each with a width of 5-7 msec for each heart beat. 2. The 5 Khz uncoded protocol 50, which includes a 5 Khz signal that is PPM modulated by a single pulse with a width of approximately 25 msec for each heart beat. 3. The ANT (now called ANT+) standard 48. 4. The Bluetooth standard 47.

The sensor proposed by the present invention also facilitates heart rate measurements from a body part which is covered by an apparel (e.g., cloth, leather etc.) or by natural fur. For example, the sensor may be integrated into a shoe and is capable of measuring the heart rate of an animal through its fur.

Calibration of the sensor can also be performed by a user pressing on their artery upstream of the location where the sensor receives signals from the artery, and then relaxing pressure. If the pressure is sufficient to cease flow of blood in the artery, then the sensor will measure a signal corresponding the zero pressure in the artery. The artery has a diameter when there is zero pressure in the artery. In the equation, above, S_{artery}(t)=α*p(t)+K, the zero pressure has “α*p(t)=0. Therefore, the S_{artery}(t) when the pressure in the artery is zero is a direct measure of “K”. The equations that model the relationship between sensed signal and arterial pressure (for example S_{artery}(t)=α*p(t)+K) and the relationship of arterial pressure versus time over some fraction of a heart beat (for example P=P0+P1*e^{-P2(t-t0)}) and a fitting of the time dependence of the sensed signal over some fraction of the heart beat (as shown for example in FIG. 7), and measure of K, enables a modeled solution for arterial pressure versus time.

1-22. (canceled)

23. A blood pressure calculation apparatus configured to calculate blood pressure of a patient based on sensing an artery pressure wave of the patient, comprising:
radar means for generating at least one radio frequency;
at least one antenna configured for positioning adjacent the skin of the patient, the at least one antenna is additionally configured to at least one of emit the at least one radio frequency into tissue of the patient and collect the reflected at least one radio frequency from the tissue;
calibration means for associating one or more sensed pressure wave values with intentional induced changes in blood pressure of the patient; and
calculation means to calculate the difference between the Systolic and Diastolic blood pressures, configured to estimate systolic and diastolic blood pressure values difference based on reflection amplitude; and
wherein said radar means is configured to transmit at a repetition rate sufficient to capture changes in the reflected at least one radio frequency throughout a heart pulse cycle.

24. The apparatus of claim 1:
wherein the calculation means receives sense signals from the radar unit corresponding to changes in artery pressure; and
wherein the calculation unit applies an algorithm to the sense signals to determine artery pressure as a function of time.

25. The apparatus of claim 1 wherein said radar means for generating at least one radio frequency is designed to generate radio frequencies between about 2 GHz and about 11 GHz.

26. The apparatus of claim 1 further comprising an article designed to be worn, and wherein: said radar means; said calibration means; and said calculation means are attached to said article.

27. The apparatus of claim 26 wherein said at least one antenna comprises printed slot antennas on a dielectric substrate.

28. The apparatus of claim 27 wherein said printed slot antennas on said dielectric substrate are positioned essentially tangential to the skin surface nearest to the dielectric substrate when said apparatus is worn.

29. A device for sensing an artery pressure wave of a mammal, comprising:
a radar unit comprising an oscillator for generating microwave signals; at least one antenna; a mixer; a low pass filter; wherein a signal generated by said oscillator is coupled to at least one of said at least one antenna and an input of said mixer; a signal received by at least one of said at least one antenna is coupled to an input of said mixer; and an output of said mixer is coupled to in input of said low pass filter;
a calculation unit comprising a signal processor, wherein an input of said calibration unit is coupled to receive sense signals derived from an output of said low pass filter;
wherein said sense signals contain information corresponding to changes in artery pressure;
wherein the calculation unit uses said signal processor to apply an algorithm to said sense signals to determine values corresponding to artery pressure as a function of time; and
wherein said radar unit is configured to transmit at a repetition rate sufficient to capture changes in the reflected at least one radio frequency throughout a heart pulse cycle.

30. The device of claim 29 wherein said algorithm comprises matching a time segment of signal associated with the artery to a model of arterial blood pressure versus time.

31. The device of claim 30 wherein said model of arterial blood pressure versus time assumes amplitude of said signal associated with the arterial pressure decays with time.

32. The device of claim 31 wherein said model of arterial blood pressure versus time assumes amplitude of said signal associated with the arterial pressure exponentially decays with time.

33. The apparatus of claim 29 wherein said radar unit is designed to generate radio frequencies over at least a range of 3 GHz.

34. The apparatus of claim 29 further comprising an article designed to be worn, and wherein said device for sensing is attached to or incorporated into said article.

35. The apparatus of claim 29 wherein said at least one antenna comprises at least one printed slot antenna on a dielectric substrate.

36. The apparatus of claim 29 further comprising an wearable article designed to be worn, and wherein said device for sensing is attached to or incorporated into said wearable article;
wherein said at least one antenna comprises at least one printed slot antenna on a dielectric substrate;
wherein said at least one printed slot antenna on said dielectric substrate is positioned essentially parallel to the skin surface of the wearer that is nearest to the dielectric substrate when said wearable article is worn.

37. The apparatus of claim 29 further comprising a wearable article designed to be worn, and wherein said device for sensing is attached to or incorporated into said wearable article; and wherein said wearable article is designed to be worn so that said device for sensing is not pressed against skin of a wearer.

38. The device of claim 29 wherein the radar unit is configured to generate microwave signals having a signal bandwidth of more than 2 GHz and less than 10.6 GHz.

39. The device of claim 29, further comprising a wrist band containing said radar unit and said calculation unit.

40. The device of claim 29 configured so that said low pass filter receives an output of said mixer; and
further comprising an IF amplifier wherein said IF amplifier receives an output of said low pass filter.

41. A method for sensing an artery pressure wave of a mammal, using a wrist wearable system comprising a radar unit comprising an oscillator for generating microwave signals; at least one antenna; a mixer; a low pass filter; wherein a signal generated by said oscillator is coupled to at least one of said at least one antenna and an input of said mixer; a reflected signal received by at least one of said at least one antenna is coupled to an input of said mixer; and an output of said mixer is coupled to in input of said low pass filter; a calculation unit comprising a signal processor, wherein an input of said calibration unit is coupled to receive sense signals derived from an output of said low pass filter; wherein said sense signals contain information corresponding to changes in artery pressure; and wherein the calculation unit uses said signal processor to apply an algorithm to said sense signals to determine values corresponding to artery pressure as a function of time; comprising:
the radar unit transmitting the generated microwave signals at a repetition rate sufficient to capture changes in the reflected signal throughout a heart pulse cycle;
coupling a signal generated by said oscillator to at least one of said at least one antenna and an input of said mixer;
coupling a signal received by at least one of said at least one antenna to an input of said mixer;
coupling an output of said mixer to in input of said low pass filter;
coupling an input of the calibration unit to receive sense signals derived from an output of said low pass filter;
wherein said sense signals contain information corresponding to changes in artery pressure;
wherein the calculation unit uses said signal processor to apply an algorithm to said sense signals to determine values corresponding to artery pressure as a function of time.

42. The method of claim 41 wherein said repetition rate is at least 30 per second and the radar unit generating microwave signals having a signal bandwidth of more than 2 GHz and less than 10.6 GHz.
